(19) United States
(12) Patent Application Publication
(10) Pub. No.: US 2008/0281182 A1
(43) Pub. Date: Nov. 13, 2008

(54) Method and apparatus for improving and/or validating 3D segmentations

(75) Inventors: Stein Inge Rabben, Sofiemyr (NO); Sevald Berg, Horten (NO); Andreas Heimdal, Oslo (NO)

Correspondence Address: DEAN D. SMALL;THE SMALL PATENT LAW GROUP LLP, 225 S. MERAMEC, STE. 725T, ST. LOUIS, MO, 63105, US

(73) Assignee: GENERAL ELECTRIC COMPANY

(21) Appl. No.: 11/800,556

(22) Filed: May 7, 2007

(51) Int. Cl. A61B 6/00

(52) U.S. Cl. 600/407

(57) ABSTRACT

A method is provided for improving a segmentation of a 3D image and/or validating a segmentation of a 3D image includes rendering an acquired 3D image and a segmentation of the acquired 3D image on a segmentation display that has at least one spatially fixed slice and an interactive slice with a reference mark corresponding to the cursor location in the spatially fixed slice or slices on the display. The method further includes utilizing an interactive user input to update image data of the interactive slice and the reference mark to coincide with the cursor in the spatially fixed slice or slices. The method further includes using the cursor and the reference mark to verify that cursor locations on the boundaries of the segmentation of the acquired 3D image correspond to object boundaries in the image data of the interactive slice.

### BACKGROUND OF THE INVENTION

This invention relates generally to methods and apparatus for improving and/or validating three-dimensional (3D) segmentation, and is particularly useful in conjunction with ultrasound image data, especially echocardiographic image data.

Automated segmentation methods are commonly used to outline objects in volumetric image data. Various methods are known that are suitable for 3D segmentation. Most of the segmentation methods rely upon deforming an elastic model towards an edge or edges in the volumetric image data. In echocardiography, it is becoming a standard clinical practice to measure 3D-based left ventricular (LV) volumes and ejection fractions (EF) from 3D segmentations.

The segmentation of noisy ultrasound data may require manually setting initial points within a region of interest (ROI) to help the segmentation algorithm identify boundaries of a segment. In some situations, it is difficult for an operator to know where to set initial points. Further, measuring wrong chamber volumes can adversely affect diagnoses or procedures to be performed on a patient.

For automated segmentation methods in 2D image data, it is often beneficial to loop through the cardiac cycle to obtain a temporal assessment of the detected contours because a boundary of an object may only be visible in a subset of the data frames. However, looping through the cardiac cycle is time-consuming because an operator has to control the looping and return to a frame that is being validated.

### BRIEF DESCRIPTION OF THE INVENTION

In one embodiment of the invention a method is provided for improving a segmentation of a 3D image and/or validating a segmentation of a 3D image. The method uses a computer having a processor, a display, a memory, and a user interface, and includes rendering an acquired 3D image and a segmentation of the acquired 3D image on a segmentation display that has at least one spatially fixed slice and an interactive slice with a reference mark corresponding to the cursor location in the spatially fixed slice or slices on the display. The method further includes utilizing an interactive user input to update image data of the interactive slice and the reference mark to coincide with the cursor in the spatially fixed slice or slices. The method further includes using the cursor and the reference mark to verify that cursor locations on the boundaries of the segmentation of the acquired 3D image correspond to object boundaries in the image data of the interactive slice.

Another embodiment of the invention provides an apparatus for improving a segmentation of a 3D image and/or validating a segmentation of a 3D image. The apparatus includes a computer having a processor, a display, memory, a user interface, and a rendering module configured to render an acquired 3D image and a segmentation of the acquired 3D image. The apparatus is configured to utilize an interactive user input to update image data of an interactive slice and a reference mark to coincide with a cursor in at least one spatially fixed slice to thereby allow a user, utilizing the cursor and the reference mark, verifying that cursor locations on boundaries of the segmentation of the acquired 3D image correspond to object boundaries in the image data of the interactive slice.

Yet another embodiment of the present invention provides a machine readable medium or media having recorded thereon instructions configured to instruct a computer having a processor, a display, memory, and a user interface. The instructions instruct the computer to segment an acquired 3D image, render an acquired 3D image and a segmentation of the acquired 3D image, display at least one spatially fixed slice and a interactive slice, and utilize an interactive user input from the user interface to update the segmentation of the acquired 3D image and the display of the spatially fixed slice or slices and the interactive slice.

### BRIEF DESCRIPTION OF THE DRAWINGS

FIG. 1 is a block diagram of an ultrasound imaging apparatus formed in accordance with various embodiments of the invention.

FIG. 2 is a pictorial view of a miniaturized ultrasound imaging apparatus formed in accordance with various embodiments of the invention.

FIG. 3 is a pictorial view of a hand-held ultrasound imaging apparatus formed in accordance with various embodiments of the invention.

FIG. 4 is a drawing illustrating resulting boundaries and a surface model of a 3D segmentation algorithm in accordance with various embodiments of the invention.

FIG. 5 is a drawing of a segmentation initialization screen of an embodiment of the invention including an interactive slice, and in which an ultrasound image to the right updates automatically according to a cursor location in an ultrasound image to the left.

FIG. 6 is a flowchart of an initialization procedure used in one embodiment of the invention.

FIG. 7 is a drawing illustrating another embodiment of a segmentation initialization screen showing three apical slices rotated around a common axis and shown together with an interactive slice.

FIG. 8 is a flowchart of a validation and editing procedure used in an embodiment of the present invention.

FIG. 9 is a drawing of a segmentation validation and editing screen formed in accordance with an embodiment of the invention.

FIG. 10 is a drawing of another embodiment of a segmentation validation and editing screen.

FIG. 11 is a drawing of yet another embodiment of the segmentation validation and editing screen.

FIG. 12 is a drawing of the segmentation validation and editing screen of FIG. 11 showing improvements made as a result of editing a segmentation.

FIG. 13 is a drawing representing a slicing plane translating around a cursor position in a spatial yoyo in accordance with various embodiments of the invention.

FIG. 14 is a drawing representing a slicing plane rotating about a common rotation axis in accordance with various embodiments of the invention, wherein the cursor position is not on the rotation axis.

FIG. 15 is a flow chart of a method used in some embodiments of the invention.

FIG. 16 is a flow chart detailing one of the steps in the flow chart of FIG. 15.

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Technical effects of various embodiments of the present invention include displaying a spatial neighborhood of a wall region in a segmentation so that an operator is able to correctly identify the object boundary.

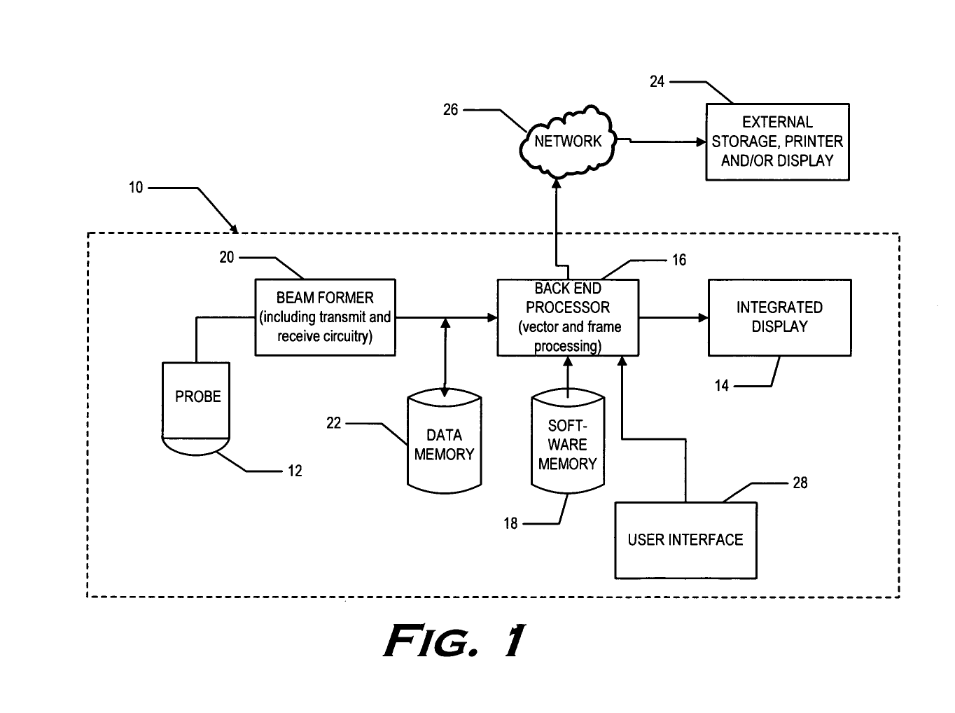

FIG. 1 is a block diagram of a medical imaging system 10 having a probe or transducer 12 configured to acquire raw medical image data. In some embodiments, probe 12 is an ultrasound transducer and medical imaging system 10 is an ultrasound imaging apparatus. A display 14 (e.g., an internal and/or integrated display) is also provided and is configured to display a medical image. A data memory 22 stores acquired image data, which has been processed by a beam former 20. The term “raw image data,” as used herein, refers to the acquired image data stored in data memory 22, which may include scan converted image data.

To display a medical image using probe 12, a back end processor 16 is provided with a software or firmware memory 18 containing instructions to perform frame processing and scan conversion using acquired raw medical image data from probe 12, possibly further processed by beam former 20. Dedicated hardware may be used instead of software and/or firmware for performing scan conversion, or a combination of dedicated hardware and software, or software in combination with a general purpose processor or a digital signal processor. Once the requirements for such software and/or hardware and/or dedicated hardware are gained from an understanding of the descriptions of embodiments of the invention contained herein, the choice of any particular implementation may be left to a hardware engineer and/or software engineer. However, for purposes of the present disclosure, any dedicated and/or special purpose hardware or special purpose processor is considered subsumed in the block labeled “back end processor 16.”

Software or firmware memory 18 can comprise a read only memory (ROM), random access memory (RAM), a miniature hard drive, a flash memory card, or any kind of device (or devices) configured to read instructions from a machine-readable medium or media. The instructions contained in software or firmware memory 18 (hereinafter referred to simply as “software memory 18”) further include instructions to produce a medical image of suitable resolution for display on display 14, to send acquired raw image data stored in a data memory 22 to an external device 24, such as a computer, and other instructions to be described below. The image data may be sent from back end processor 16 to external device 24 via a wired or wireless network 26 (or direct connection, for example, via a serial or parallel cable or USB port) under control of back end processor 16 and user interface 28. In some embodiments, external device 24 may be a computer or a workstation having a display and memory. User interface 28 (which may also include display 14) also receives data from a user and supplies the data to back end processor 16. In some embodiments, display 14 may include an x-y input, such as a touch-sensitive surface and a stylus (not shown), to facilitate user input of data points and locations. The initialization of the segmentation module, the segmentation, the validation of the segmentation and the editing of segmentation is also done by the instructions stored in software memory 18.

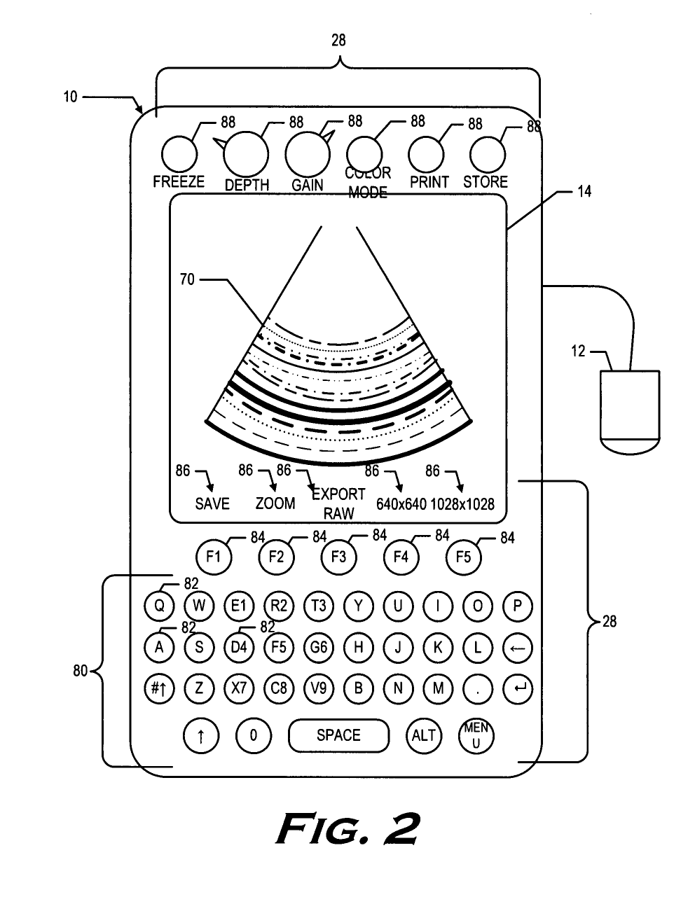

FIG. 2 is a pictorial drawing of an embodiment of medical imaging system 10 configured as a hand carried device. Medical imaging system 10 includes display 14, for example, a color LCD touch-sensitive display (on which a medical image 70 may be displayed) and the user interface 28. In some embodiments of the present invention, a typewriter-like keyboard 80 of buttons 82 is included in user interface 28, as well as one or more soft keys 84 that may be assigned functions in accordance with the mode of operation of medical imaging system 10. A portion of display 14 may be devoted to labels 86 for soft keys 84. For example, the labels shown in FIG. 2 allow a user to save the current raw medical image data, to zoom in on a section of image 70 on display 14, to export raw medical image data to an external device 24 (shown in FIG. 1), or to display (or export) an image. The device may also have additional keys and/or controls 88 for special purpose functions.

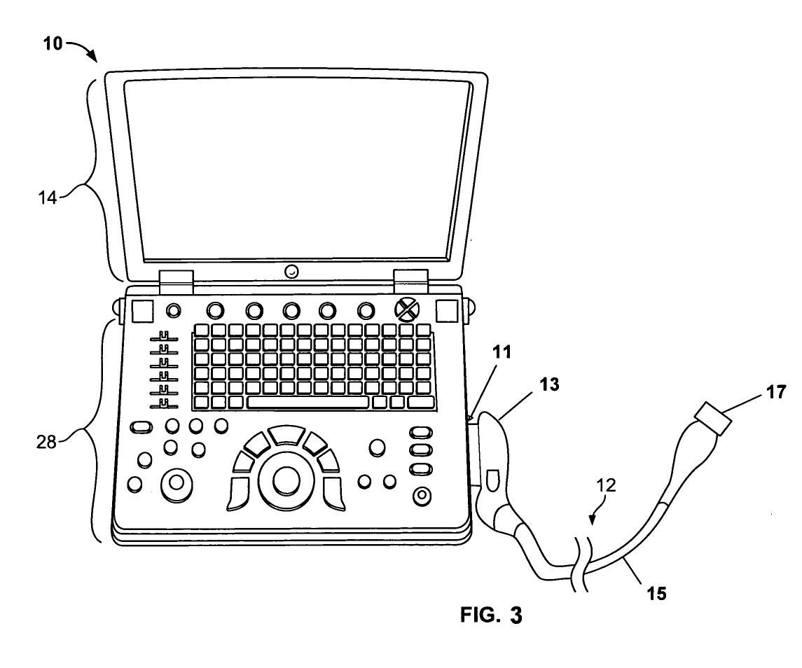

FIG. 3 illustrates a medical imaging system 10 configured as a miniaturized ultrasound device. As used herein, “miniaturized” means that medical imaging system 10 is a handheld or hand-carried device or is configured to be carried in a person's hand, briefcase-sized case, or backpack. For example, medical imaging system 10 may be a hand-carried device having a size of a typical laptop computer

An ultrasound probe 12 has a connector end 13 that interfaces with medical imaging system 10 through an I/O port 11 on medical imaging system 10. Probe 12 has a cable 15 that connects connector end 13 and a scanning end 17 that is used to scan a patient. Medical imaging system 10 also includes display 14 and user interface 28.

Embodiments of the present invention can comprise software or firmware instructing a computer to perform certain actions. Some embodiments of the present invention comprise stand-alone workstation computers that include memory, a display, and a user input interface (which may include, for example, a mouse, a touch screen and stylus, a keyboard with cursor keys, or combinations thereof). The memory may include, for example, random access memory (RAM), flash memory, and read-only memory. For purposes of simplicity, devices that can read and/or write media on which computer programs are recorded are also included within the scope of the term “memory.” A non-exhaustive list of media that can be read with a suitable such device includes CDs, CD-RWs, DVDs of all types, magnetic media (including floppy disks, tape, and hard drives), flash memory in the form of sticks, cards, and other forms, ROMs, etc., and combinations thereof.

Some embodiments of the present invention may be incorporated into a medical imaging apparatus, such as medical imaging system 10 of FIG. 1. In correspondence with a stand-alone workstation, the “computer” is the medical imaging system 10. For example, back end processor 16 may comprise a general purpose processor with memory, or a separate processor and/or memory may be provided. Display 14 corresponds to the display of the workstation, while user interface 28 corresponds to the user interface of the workstation. Whether a stand-alone workstation or an imaging apparatus is used, software and/or firmware (hereinafter referred to generically as “software”) are used to instruct the computer to perform the inventive combination of actions described herein. Portions of the software may have specific functions, and these portions are herein referred to as “modules” or “software modules.” However, embodiments of the present invention are not limited to being implemented in software module. Thus, the term “module” is also intended to encompass functions that are partly or completely implemented in hardware, with or without the use of software or firmware.

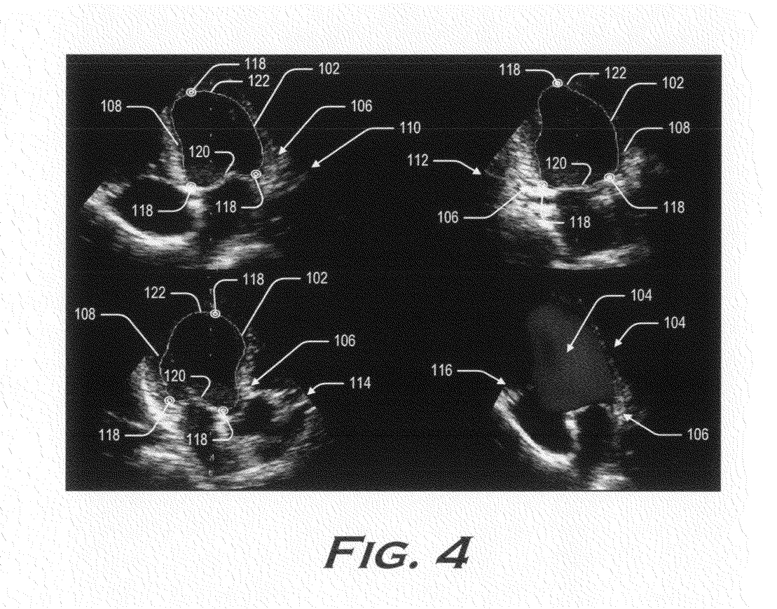

Some embodiments of the present invention provide a segmentation algorithm for volumetric image data, while other embodiments use a pre-existing segmentation. FIG. 4 is an illustration of the segmentation of boundaries 102 and a surface model 104 in a volumetric object 106 using a 3D segmentation algorithm, in one embodiment in which a segmentation algorithm is provided. The segmentation algorithm detects boundaries 108 of volumetric object 106. In the example represented in FIG. 1, volumetric object 106 is a human heart, and boundaries 108 are the inner walls of the left ventricle of the heart. Most segmentation algorithms have in common that boundaries 102 of an elastic model deform towards edges 108 in volumetric data. The illustrated algorithm segments the volumetric object 106 within the volumetric data. Volumetric object 106 together with slices 110, 112, 114, and 116 of the image data are then displayed by a renderer in segmentation initialization, and validation and editing screens on a display, such as display 14.

Small round circles 118 in FIG. 4 shown around a valve 120 and also at an apex 122 at the upper part of each image slice 110, 112, and 114 represent an initial region of interest for the segmentation algorithm. A technical effect of some embodiments of the present invention is the providing of methods and/or apparatus for performing the initial guess or estimate and to display the results of segmentation. If the segmentation algorithm did not work properly or not satisfactory, another technical effect of some embodiments of the present invention is to provide methods and apparatus for editing the segmentation by positioning attractors.

When an operator initializes or edits a segmentation, it is important for the operator to confirm that the cursor is actually located on a wall boundary. However, ultrasound data may contain image artifacts such as reverberations and dropouts. As a result, when an operator inspects a single slice view intersecting a 3D model and the image data, it may be difficult for the operator to visually identify the exact location of the object boundary. Also, when the object boundary is almost parallel to the slice plane, it may be difficult to select the correct location for initial or edit points.

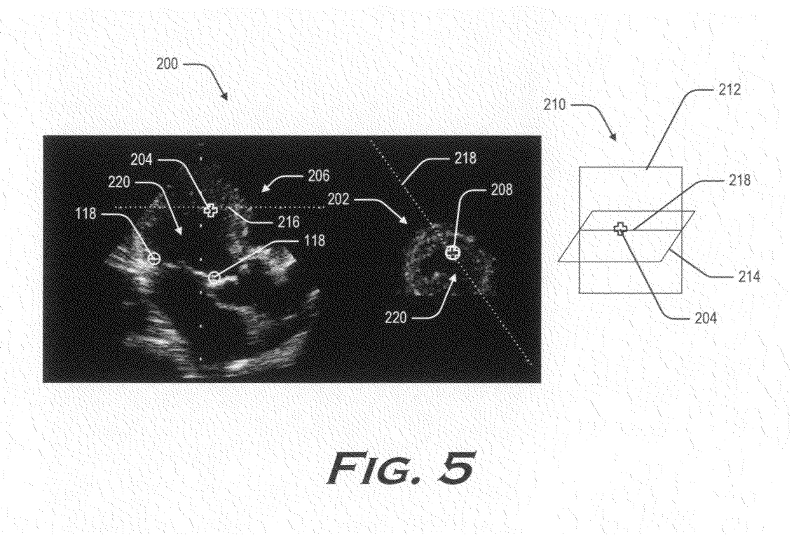

A drawing of one embodiment of an interactive slicing display 200 is shown in FIG. 5. Interactive slice 202 (which acts as a “slave”) updates automatically according to the location of cursor 204 in spatially fixed slice 206 (which acts as the “master”). Thus, when cursor 204 is moved in spatially fixed slice 206, a reference mark 208 moves in interactive slice 202 to a location corresponding to the position of cursor 204. As seen in inset 210 (which is not necessarily part of interactive slicing display 200), spatially fixed slice 206 is located in one plane 212, whereas interactive slice 202 is located in a plane 214 that is perpendicular to plane 212. An intersection line 218 may be indicated in interactive slice 202 for purposes of aiding the positioning of spatially fixed slice 206. Also, an intersection line 216 in spatially fixed slice 206 may be indicated to show the location of interactive slice 202. Interactive slicing display 200 may be used for manually positioning initial points 118 for a segmentation algorithm, or for validating or editing the segmentation results.

More generally, some embodiments of the present invention provide an interactive slicing display 200 such as that shown in FIG. 5. A master-slave relation between a spatially fixed slicing plane 212 under the cursor in a master display 206 and the interactive slice 202 assures that an operator is able to see a region of interest indicated generally at 220. Slice plane 214 of interactive slice 202 includes the location of cursor 204 in three-dimensional (3D) space, but does not coincide with master slice plane 212. A slicing plane 214 that is orthogonal to master slice plane 212 may be useful, but orthogonality of planes 212 and 214 is not required in embodiments of the present invention. When an operator moves cursor 204 within the spatially fixed slicing plane 212, interactive slice 202 and reference mark 208 update accordingly.

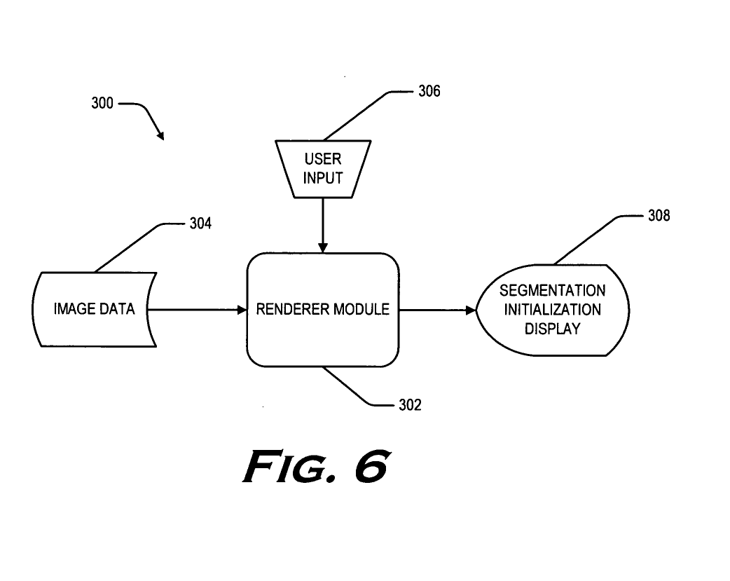

FIG. 6 is a flowchart 300 of an initialization procedure used in an embodiment of the present invention. A renderer module 302 is used to display image data 304 in a segmentation initialization display 308, which is, for example, interactive slicing display 200. Not all segmentation methods require manual input of initial points and/or a region of interest, and thus, do not require an initialization screen 308. However, for those segmentation methods that do require a manual user input 306, this user input is obtained from the user while the segmentation initialization screen 308 is displayed.

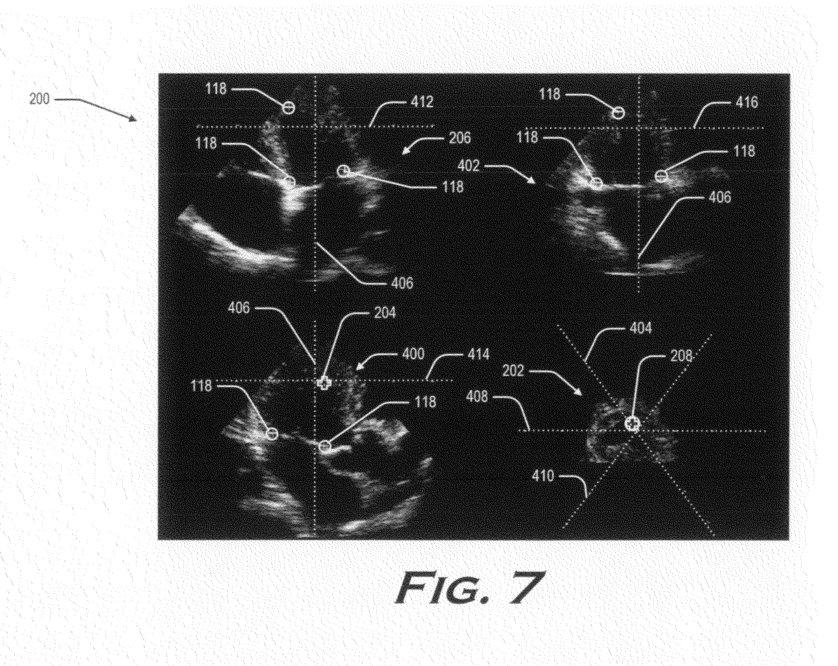

In some embodiments in which the image is, for example, an echocardiographic image of a heart, an apical slice can be used as master image 206. However, as shown in FIG. 7, a plurality of apical slices 206, 400 and 402 can be displayed in an interactive slicing display 200 along with an interactive slice 202. The display of a plurality of apical slices 206, 400, and 402 can be used to more fully visualize the whole object for selecting points to initialize a segmentation. Cursor 204 can be in any of the three slices 206, 400, or 402. In FIG. 4, cursor 204 is in slice 400, and thus a reference mark 208 corresponding to the location of cursor 204 also appears in interactive slice 202 on an intersection line 404. Intersection line 404 corresponds to slice 400 and is superimposed on interactive slice 202. Intersection line 406 is a common axis for all apical slices 206, 400, and 402. Other intersection lines 408 and 410 correspond to slices 206 and 402, respectively. Intersection lines 412, 414, and 416 correspond to the intersection of the planes containing apical slices 206, 400, and 402, respectively, with interactive slice 202.

In some embodiments of the invention, a user input is used to position a plurality of initial points 118 in a plurality of spatially fixed slices, such as apical slices 206, 400, and 402. Any number of initial points 118 may be selected, and subsets of different numbers of points may be distributed as needed across the plurality of slices 206, 400, and 402. However, in 3D images, it is sometimes difficult to know whether or not the initial points 118 are on an object boundary. Interactive slice 202 provides visible assistance in determining whether initial points 118 are actually on an object boundary. If cursor 204 is moved, the depiction of interactive slice 202 may change. Thus, some embodiments of the present invention provide a method and apparatus for setting initial points within a volume.

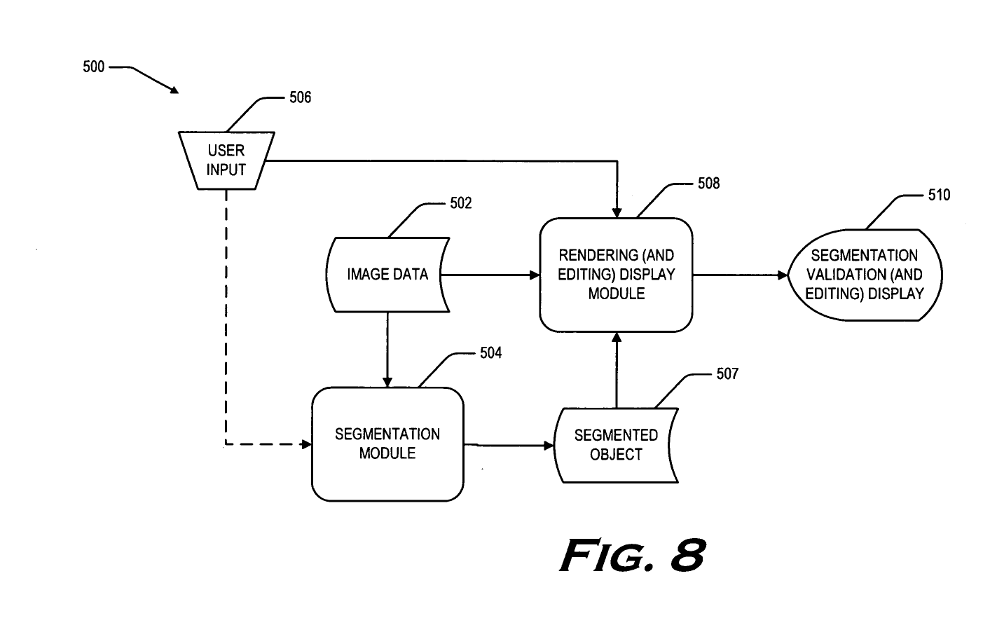

FIG. 8 is a flowchart 500 of a validation and editing procedure used in an embodiment of the present invention. Image data 502 is provided to a segmentation module 504 that uses image data 502 (with user input 506 in some embodiments, as discussed above) to generate a segmented object 507. Segmented object 507 along with image data 502 is used by a rendering module 508 to generate a segmentation validation and editing display 510. The operator uses segmentation validation and editing display 510 to provide additional input 506 to renderer module 508 to update segmentation validation and editing display 510. When the operator is satisfied with the editing that is performed by the operator, the additional input 506 (e.g., the coordinates of the revised initial or additional edit point) is provided to segmentation module 504 to update the segmented object 507. There are no restrictions on the type of segmentation algorithm used in segmentation module 504; however, the deformable model is one example for use in module 504. It should be understood that it is not a requirement in all embodiments of the present invention that the segmentation object be edited. Embodiments that do not allow or require that the segmentation object be edited also fall within the scope of the present invention. Thus, unless otherwise explicitly stated, the scope of the term “validation and editing display,” as used herein, also includes embodiments having validation displays without editing capability.

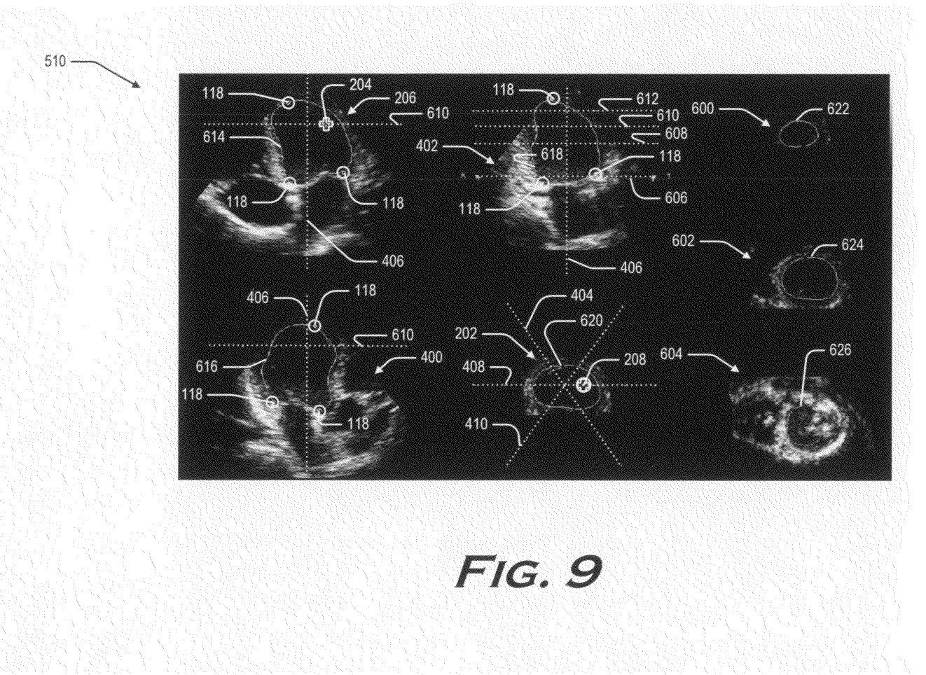

FIG. 9 is a drawing of an embodiment of a segmentation validation and editing screen display 510. This particular embodiment shows a plurality of apical slices 206, 400, and 402 as seen earlier, and a vertical axis 406 that is a rotation axis or common axis for apical slices 206, 400, and 402. An interactive slice 202 is also shown, as are a plurality of short axis (SAX) slices 600, 602, and 604. Short axis slices 600, 602, and 604 are orthogonal to apical slices 206, 400, and 402, respectively. Upper middle image 402 has four horizontal lines 606, 608, 610, and 612, three of which (606, 608, and 612) show the location or positioning of short axis slices 600, 602, and 604 on the right. Because a relatively large number of slices are presented, it is very easy to visually determine whether or not a segmentation algorithm fails. Lines 614, 616, 618, 620, 622, 624, and 626 show the result (the boundary) of the segmentation algorithm and these lines are superimposed on the grayscale image data, making the segmentation results particularly easy to see and validate.

Segmentation validation and editing display screen 510 provides the ability to edit the segmentation in some embodiments of the present invention. Cursor 204 is shown in a master slice 206. The location of cursor 204 is also indicated in interactive slice 202. By providing the cursor 204 position in an interactively updated, orthogonal slice such as interactive slice 202, in which reference mark 208 is updated to correspond to location of cursor 204, it is possible to see a boundary in a direction different from that of a master slice. Thus, it is possible to identify whether the cursor is on a boundary or not and whether the cursor has to be moved to more closely approach a boundary.

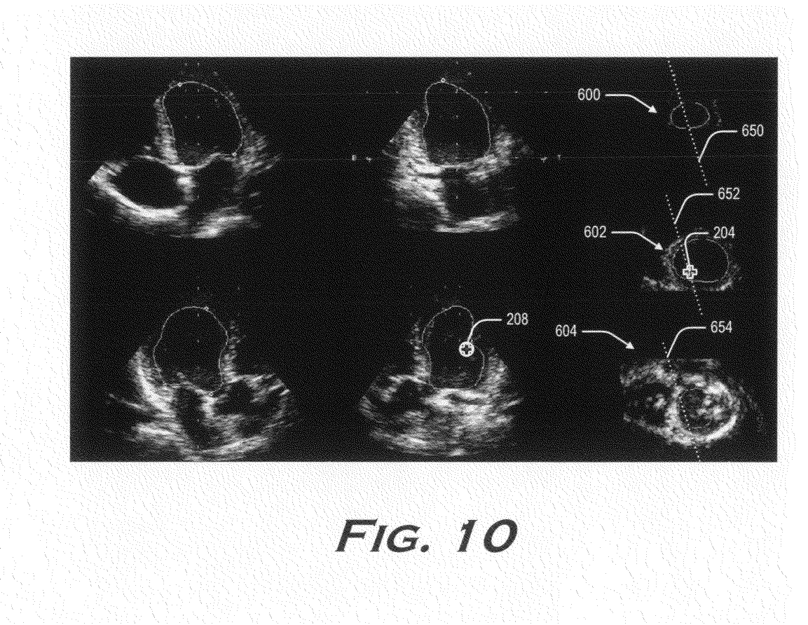

FIG. 10 is a drawing of another embodiment of a segmentation validation and editing screen 510. In this embodiment, the operator has moved cursor 204 to a short axis slice 602. As a result, interactive slice 202 has changed to an apical slicing plane intersecting the 3D location of cursor 204. Lines 650, 652, and 654 are indicative of the orientation of the interactive slice.

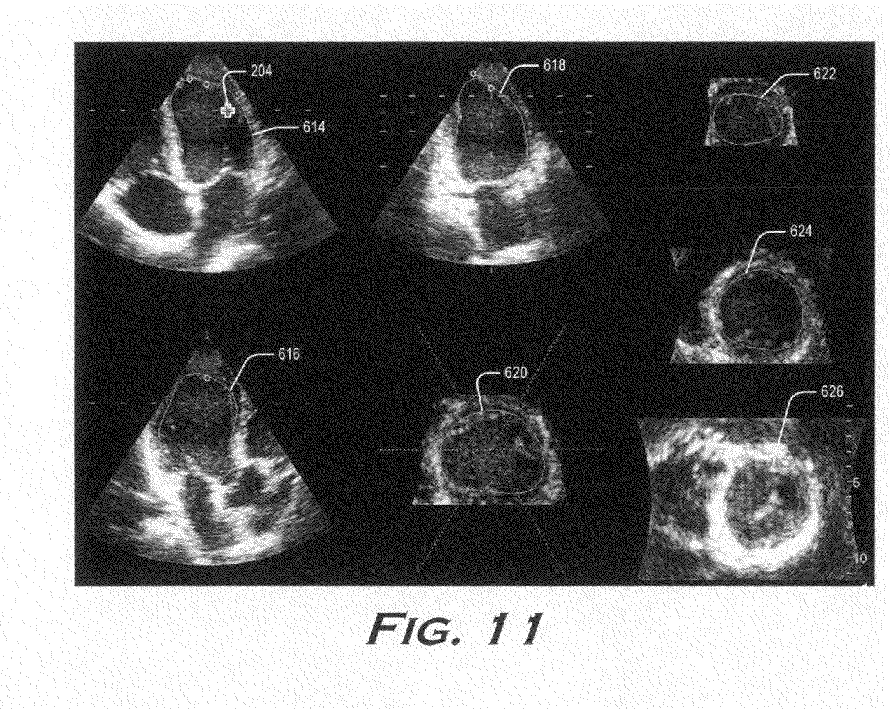

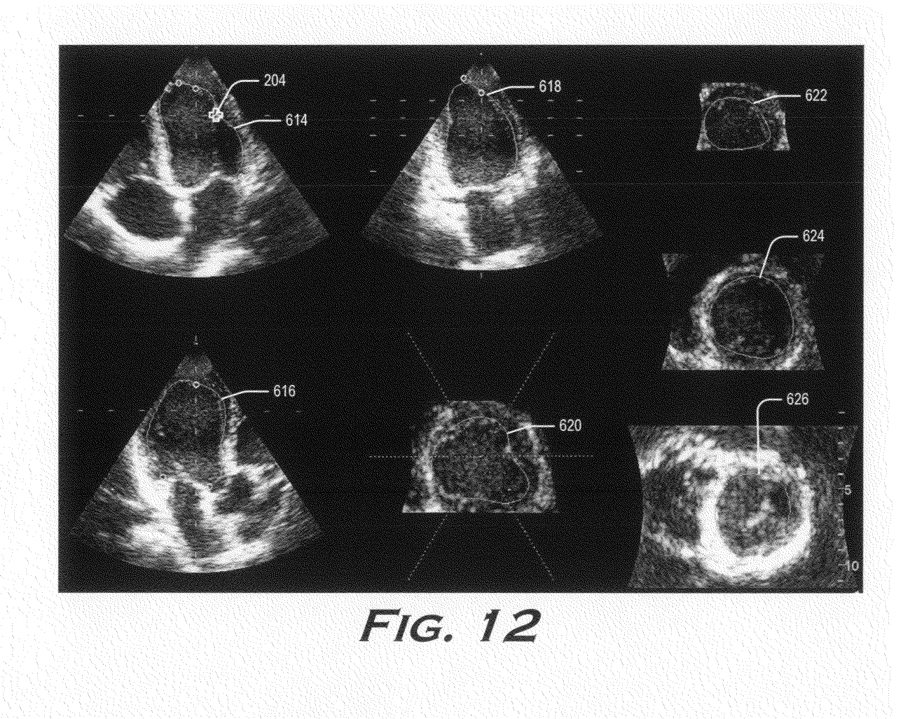

FIG. 11 is a drawing of another embodiment of a segmentation validation and editing screen 510, and FIG. 12 is a drawing of the segmentation validation and editing screen 510 of FIG. 11, showing changes made as a result of editing the segmentation in FIG. 11. Note that segment boundary lines 614, 616, 618, 620, 622, 624, and 626 have changed between FIG. 11 and FIG. 12 as a result of setting an edit point at the location of cursor 204 off of a location on line 614.

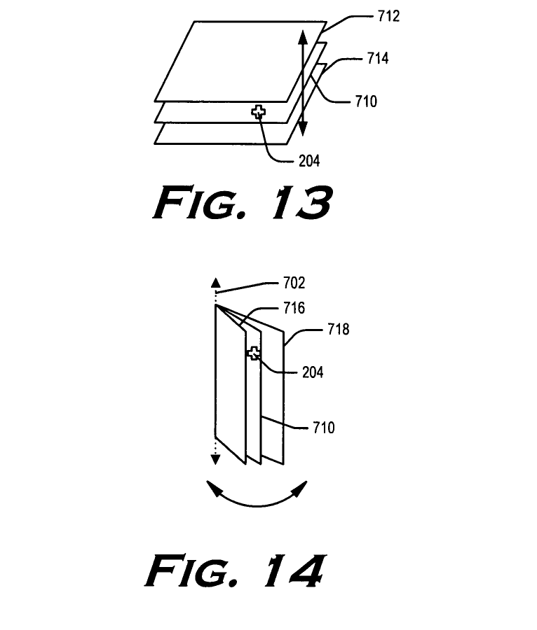

FIG. 13 is a drawing representing a slicing plane 710 translating around a cursor 204 position in a spatial yoyo. This form of spatial yoyo operates by moving a slicing plane 710 very slowly up and down between positions such as indicated by planes 712 and 714 parallel to slicing plane 710, and, in some embodiments, other parallel planes between planes 712 and 714, allowing a boundary that may not be visible on a slicing plane, but which may be visible on a different nearby plane, to be located.

FIG. 14 is a drawing representing a slicing plane 710 rotating about a common rotation axis 702, again, in a spatial yoyo, but wherein the cursor 204 position is not on rotation axis 702. This form of spatial yoyo operates by tilting a slicing plane 710 slowly back and forth between positions such as those indicated by planes 716 and 718.

Spatial yoyos may be used to locate boundaries in ultrasound images, and thus, may be included in renderers in some embodiments of the invention. More particularly, boundaries in an ultrasound image may show up only temporarily. For example, when a heart is fully contracted, the boundaries of a chamber of the heart may be readily visible, whereas at another time, the boundary may disappear or become less visible. A spatial yoyo of either or both of the types shown in FIGS. 13 and 14 allows an operator to slowly scroll back and forth when setting the initial points. The spatial yoyo operates by moving a slicing plane very slowly up and down, or by tilting the slicing plane slowly back and forth, allowing a boundary that may not be visible on a slicing plane, but which may be visible on a different nearby plane, to be located.

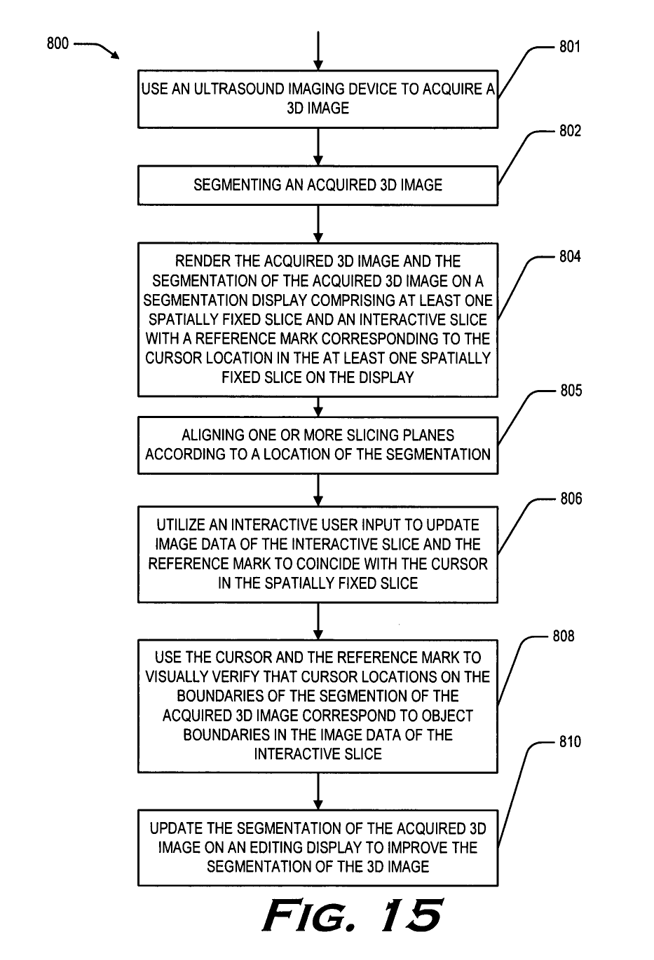

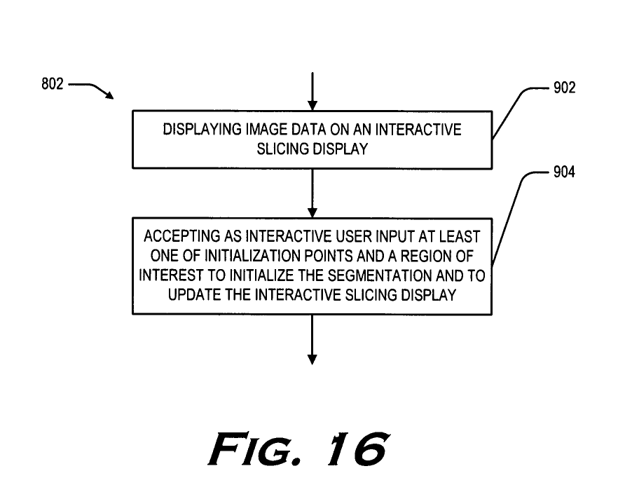

FIG. 15 is a flow chart 800 of a method used by some embodiments of the invention. The method for segmenting and validating a 3D image may use a computer 10 having back end processor 16, memory 18, and user interface 28. The method includes, at 804, rendering an acquired 3D image and a segmentation of the acquired 3D image on a segmentation display comprising at least one spatially fixed slice and an interactive slice with a reference mark corresponding to the cursor location in the at least one spatially fixed slice on the display. Next, the method further includes, at 806, utilizing an interactive user input to update image data of the interactive slice and the reference mark to coincide with the cursor in the at least one spatially fixed slice. Next, the method includes, at 808, using the cursor and the reference mark, visually verifying that cursor locations on the boundaries of the segmentation of the acquired 3D image correspond to object boundaries in the image data of the interactive slice.

In some embodiments, the method further includes, at 810, updating the segmentation of the acquired 3D image on an editing display to improve the segmentation of the 3D image. Also, in some embodiments, the method includes, at 802, segmenting the acquired 3D image. FIG. 16 is a flowchart detailing steps included in some embodiments of the present invention in box 802. For example, segmenting the acquired 3D image may comprise, at 902, displaying image data on an interactive slicing display and, at 904, accepting as interactive user input at least one of initialization points and a region of interest to initialize the segmentation and to update the interactive slicing display. Furthermore, block 902 may further comprise displaying a plurality of short axis slices of the region of interest located along the common axis of the apical slices.

Returning to FIG. 15, in some embodiments of the present invention, rendering the acquired 3D image at 804 may comprise displaying a plurality of spatially fixed slices of a region of interest rotated around a common axis together with an interactive slicing display of the region of interest oriented around a different axis.

Some embodiments of the present invention include, at 805, aligning one or more slicing planes according to a location of the segmentation. Also, in some embodiments, block 806 may include at least one of translating and rotating a slicing plane to facilitate visibility of an object of interest in the image data and selection of the interactive user input to update the segmentation of the acquired 3D image. In some embodiments, the method also includes, at 801, using an ultrasound imaging device to acquire the 3D image. The acquired ultrasound 3D image can include an image of a heart of a patient, and the segmentation can comprise segmenting the heart of the patient.

It will thus be appreciated that some embodiments of the present invention provide an interactive method and apparatus to initialize and/or validate and edit a segmentation. Also, some embodiments provide more reliable initialization, validation and editing of a segmentation, as well as more reproducible end-results, most notably volume measurements of segments in an object.

Also, it will be appreciated that some embodiments of the invention provide methods and apparatus for revealing where a boundary exists in volumetric image data, to improve the visual assessment of where the true object boundary is in an image by observing the spatial neighborhood of a contour under inspection.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. § 112, sixth paragraph, unless and until such claim limitations expressly use the phrase “means for” followed by a statement of function void of further structure.

What is claimed is:

1. A method for at least one of improving a segmentation of a 3D image or validating a segmentation of a 3D image, said method using a computer having a processor, a display, a memory, and a user interface, and said method comprising:
rendering an acquired 3D image and a segmentation of the acquired 3D image on a segmentation display comprising at least one spatially fixed slice and an interactive slice with a reference mark corresponding to the cursor location in the at least one spatially fixed slice on the display;
utilizing an interactive user input to update image data of the interactive slice and the reference mark to coincide with the cursor in the at least one spatially fixed slice; and
using the cursor and the reference mark, to verify that cursor locations on the boundaries of the segmentation of the acquired 3D image correspond to object boundaries in the image data of the interactive slice.

2. A method in accordance with claim 1 further comprising updating the segmentation of the acquired 3D image on an editing display to improve the segmentation of the 3D image.

3. A method in accordance with claim 1 further comprising segmenting the acquired 3D image, and said segmenting the acquired 3D image comprises displaying image data on an interactive slicing display and accepting as interactive user input at least one of initialization points and a region of interest to initialize the segmentation and to update the interactive slicing display.

4. A method in accordance with claim 1 wherein rendering the acquired 3D image data comprises displaying a plurality of spatially fixed slices of a region of interest rotated around a common axis together with an interactive slicing display of the region of interest oriented around a different axis.

5. A method in accordance with claim 4 wherein displaying image data on the interactive slicing display further comprises displaying a plurality of short axis slices of the region of interest located along the common axis of the apical slices.

6. A method in accordance with claim 5 further comprising updating locations of the plurality of short axis slices.

7. A method in accordance with claim 4 further comprising aligning one or more slicing planes according to a location of the segmentation.

8. A method in accordance with claim 1 further comprising at least one of translating and rotating a slicing plane of the interactive slice to facilitate visibility of an object of interest in the image data.

9. A method in accordance with claim 1 wherein the verifying comprises visually verifying.

10. A method in accordance with claim 1 wherein the acquired ultrasound 3D image includes an image of a heart of a patient, and the segmentation comprises segmenting the heart of the patient.

11. An apparatus for at least one of improving a segmentation of a 3D image or validating a segmentation of a 3D image, said apparatus comprising:
a computer having a processor, a display, memory, and a user interface;
a rendering module configured to render an acquired 3D image and a segmentation of the acquired 3D image; and
said apparatus configured to utilize an interactive user input to update image data of an interactive slice and a reference mark to coincide with a cursor in at least one spatially fixed slice to allow utilizing the cursor and the reference mark, verifying that cursor locations on boundaries of the segmentation of the acquired 3D image correspond to object boundaries in the image data of the interactive slice.

12. An apparatus in accordance with claim 11 wherein to aid a user in segmenting the acquired 3D image, said apparatus further comprises a segmentation module configured to display image data on an interactive slicing display and to receive an interactive user input comprising at least one of initialization points and a region of interest to initialize the segmentation and to update the interactive slicing display.

13. An apparatus in accordance with claim 12 wherein to display image data on an interactive slicing display, said apparatus further comprises an editing display module configured to display a plurality of spatially fixed slices of a region of interest rotated around a common axis together with an interactive slice displaying the region of interest oriented around a different axis.

14. An apparatus in accordance with claim 13 wherein to display image data on an interactive slicing display, the editing display module is further configured to display a plurality of short axis slices of the region of interest located along the common axis of the spatially fixed slices.

15. An apparatus in accordance with claim 14 wherein the rendering module is further configured to update locations of the plurality of short axis slices after said updating of said segmentation is performed.

16. An apparatus in accordance with claim 13 wherein the rendering module is further configured to align one or more slicing planes according to a location of the segmentation.

17. An apparatus in accordance with claim 13 further comprising a spatial yoyo module configured to instruct the computer to at least one of translate and rotate a slicing plane to facilitate visibility of an object of interest in the image data and selection of the interactive user input to update the segmentation of the acquired 3D image.

18. An apparatus in accordance with claim 11 further comprising an ultrasound probe and a beam former with transmit and receive circuitry configured to acquire ultrasound 3D image data.

19. A machine readable medium or media having recorded thereon instructions configured to instruct a computer having a processor, a display, memory, and a user interface to:
render an acquired 3D image and a segmentation of the acquired 3D image;
display at least one spatially fixed slice and a interactive slice; and
utilize an interactive user input from the user interface to update the segmentation of the acquired 3D image and the display of the at least one spatially fixed slice and the interactive slice.

20. A medium or media in accordance with claim 19, wherein said instructions further configured to instruct the computer to segment an acquired 3D image, and wherein said instructions to segment the acquired 3D image include instructions to display image data on an interactive slicing display and receive an interactive user input comprising at least one of initialization points and a region of interest to initialize the segmentation and to update the interactive slicing display.
